(19) United States
(12) Patent Application Publication
(10) Pub. No.: US 2004/0110653 A1
(43) Pub. Date: Jun. 10, 2004

(54) Economical soap bar element

(76) Inventor: Robert Cushman Brown, Tallahassee, FL (US)

Correspondence Address: Robert C. Brown, 1207 Sandhurst Drive, Tallahassee, FL, 32312, US

(21) Appl. No.: 10/314,766

(22) Filed: Dec. 9, 2002

(51) Int. Cl. A61K 7/50

(52) U.S. Cl. 510/141; 510/147

(57) ABSTRACT

A bar element intended for personal washing use only once or a few times, after which minimal soap remains on the element. The soap bar element includes an inexpensive, preferably inert, core supporting a relatively thin shell of soap. The core may be formed of any convenient material, such as rigid or foam polyurethane or polystyrene, or of water-soluble or biodegradable material such as oxycellulose polymers. The soap may be any solid soap material. The core may extend through one or more surfaces of the soap shell. The core comprises at least about 25%, and preferably greater than about 80%, by volume of the bar element.

### TECHNICAL FIELD

The present invention relates to soaps comprising solid water-miscible cleaning materials formed in the shape of bars; more particularly, to soap bars intended for single- or limited-life use in public facilities such as hotels and motels; and most particularly, to an economical soap bar element wherein a relatively large percentage of the bar volume comprises an inexpensive non-soap core.

### BACKGROUND OF THE INVENTION

Soap bars for personal washing use are well known. Typically, a soap bar is formed of a solid water-soluble or water-miscible organic material and is homogeneous throughout. Such bars are readily cast from molten soap in known fashion.

As a soap bar is repeatedly used, surface soap is washed away and the bar becomes progressively smaller. A smaller, thinner soap bar is more difficult to use, and significant prior art is devoted to schemes for improving the use of the inner portions of a soap bar.

For example, U.S. Pat. No. 3,969,256, issued Jul. 13, 1976, discloses a soap bar having a rigid, hollow, plastic core to prevent the soap bar from breaking in half or breaking up as soap is progressively removed, and to thus permit use of the bar until all of the soap is consumed.

For a further example, U.S. Pat. No. 5,221,506, issued Jun. 22, 1993, discloses a soap bar for personal use having a structural center selected from an open-celled sponge material, or woven or non-woven organic filamentary material which is fully impregnated with soap. The sponge core is revealed after the soap bar is reduced to a sliver, providing support, preventing breakage, and making washing more effective, and reducing wastage from the typically residual sliver of soap.

Other examples of prior art disclose cores comprising prizes, scrubbing pads, and means for hanging a soap bar. The underlying objective of the prior art schemes is to support the soap in such a way as to provide complete utilization of all of the soap through repeated and long-term use of the bar.

However, another problem exists in the use of bar soap, which problem is neither addressed nor solved by the prior art. In public accommodations such as hotels and motels, small bars of soap customarily are provided for the use of overnight guests. In some instances, separate bars are provided for facial soap and general bath soap. In a great majority of such uses, such a soap bar is used only once or twice by an overnight guest and is then left behind for removal and discard by a chambermaid. The amount of the bar actually used by a guest in one or a few washings may be as little as five or ten percent, the remainder constituting complete waste of the relatively expensive soap materials.

One approach to reducing such waste is to make the individual bars smaller so that a greater percentage of the soap is used. However, smaller bars are more difficult to handle, may be perceived as cheap, and thus may not be well-received by a hotel's clientele.

What is needed is an economical soap bar of conventional size wherein a relatively small amount of soap is residual waste after relatively few uses.

It is a principal object of the present invention to reduce the cost of manufacture of a limited-use soap bar.

It is a further object of the invention to reduce the amount of bar soap wasted in hotel and motel usage.

### SUMMARY OF THE INVENTION

Briefly described, a soap bar element intended for personal washing use only one or a few times includes an inexpensive, preferably inert, bar core supporting a relatively thin shell of soap. The core may be formed of any convenient material, such as rigid or foam polyurethane or polystyrene, or of water-soluble or biodegradable material, such as but not limited to oxycellulose polymers or rigid polyvinlyalcolhol foam. The soap may be any solid soap material. The core may extend through one or more surfaces of the soap shell, for example, the ends, as occurs when individual bars are cut from a long, continuous bar. The core comprises at least about 25%, and preferably greater than about 80%, by volume of the bar element.

### BRIEF DESCRIPTION OF THE DRAWINGS

These and other features and advantages of the invention will be more fully understood and appreciated from the following description of certain exemplary embodiments of the invention taken together with the accompanying drawings, in which:

FIG. 1 is an elevational cross-sectional view of a first embodiment of a soap bar element in accordance with the invention, taken along line 1-1 in FIG. 2;

FIG. 2 is an isometric view of a first embodiment of a soap bar element in accordance with the invention, showing a core element fully encapsulated by a soap shell; and

FIG. 3 is an isometric view, partially in cross-section, of a second embodiment, showing a continuous bar of co-extruded core material and soap material from which individual bar elements may be severed.

### DESCRIPTION OF THE PREFERRED EMBODIMENTS

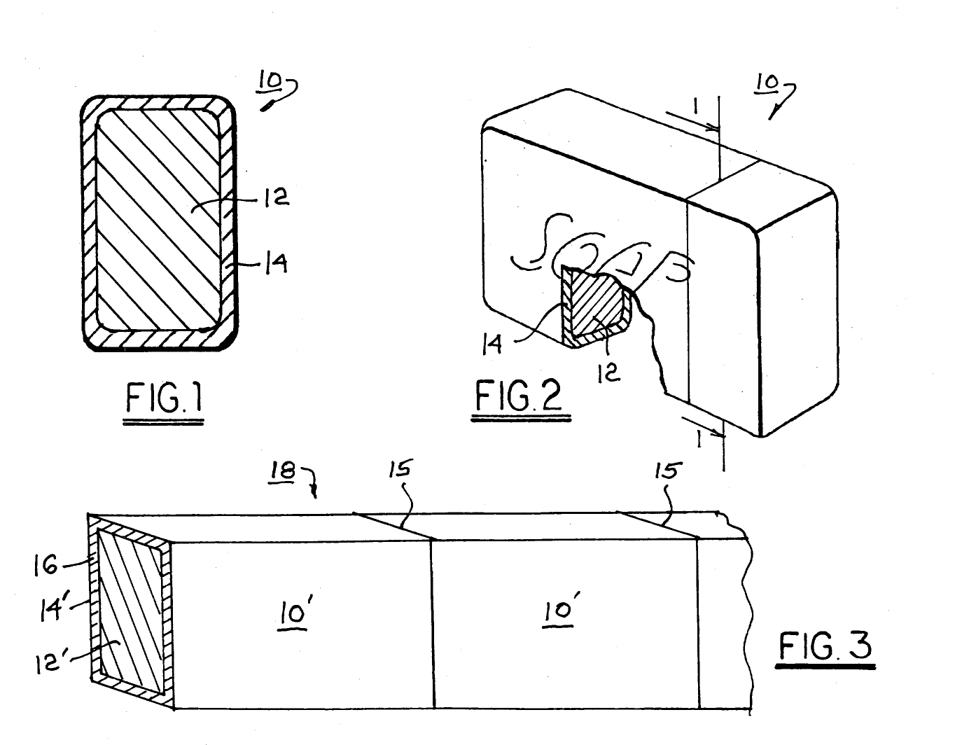

Referring to FIGS. 1 and 2, a bar element 10 in accordance with the invention, herein referred to as a soap bar, includes a core portion 12 supporting a soap shell 14 substantially surrounding core 12 in three dimensions. Core 12 may be formed of any convenient and inexpensive non-soap material, such as rigid polyurethane or polystyrene solid or foam, or of water-soluble or biodegradable material, such as but not limited to oxycellulose polymers or polyvinylalcohol foam. Core 12 may be solid or hollow and may be buoyant or not. The soap forming shell 14 may be any solid soap material such as, but not limited to, shop or lavatory hand soap, deodorant soap, antibacterial soap, bath soap, hair soap, facial soap, cosmetic soap, or any combinations thereof. Preferably, core 12 is not porous, in order that bar element 10 be formed with a minimum amount of soap material. Bar 10 may be formed by known methods, as, for example, by insertion molding of molten soap around core 12 in a mold or by dipping core 12 one or more times in molten soap, as is fully disclosed in U.S. Pat. No. 5,221,506, which patent is hereby incorporated by reference.

Referring to FIG. 3, the core may extend through one or more outer surfaces 16 of soap shell 14′ as may occur when soap bars 10′ are formed as by severing individual bar elements 10′ at locations 15 from a continuous ribbon 18. Bars 10′ do not have soap covering the exposed ends of core 12, and thus soap surrounds core 12 in only two dimensions. Ribbon 18 may be formed by co-extrusion of core material 12′ and soap material 14′, or by continuous coating of core material 12′ by molten soap material 14′, in known fashion.

A single use of soap bar 10 to wash the face or hands may remove less than about 0.5 mm of soap material. Thus, a shell thickness of 1 mm is typically sufficient for three or four washings. In such a bar having, for example, an overall starting length of 60 mm, width of 40 mm and height of 10 mm, core 12 comprises 74% of the total volume of bar 10, or 80% of bar 10′. When the starting shell thickness is reduced to 0.5 mm, core 12 comprises 86% of the total volume of bar 10, or 90% of bar 10′. Conversely, when core 12 comprises, for example, 50% of the volume of bar 10, the starting thickness of shell 14 is more than 2 mm. When core 12 comprises 25% of the volume of bar 10, the starting thickness of shell 14 is more than 3 mm.

Soap bars having other starting dimensions may be similarly configured within the scope of the invention.

In each case, core 12 replaces a significant volume of soap used to form a bar in accordance with the prior art. Further, in each case a relatively small amount of soap remains as waste after one or a few uses of the bar. Thus, very significant savings in soap volume may be achieved in accordance with the invention, as desired for any specific application, without compromising either washing performance for up to several uses or desirable bar size.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed is:

1. A bar element for personal washing, comprising a central core and a soap shell surrounding said core in at least two dimensions, wherein said core occupies at least about 25 volume percent of said bar element.

2. A bar element in accordance with claim 1 wherein said core occupies at least about 50 volume percent of said bar element.

3. A bar element in accordance with claim 1 wherein said core occupies at least about 75 volume percent of said bar element.

4. A bar element in accordance with claim 1 wherein said core occupies at least about 90 volume percent of said bar element.

5. A bar element in accordance with claim 1 wherein said core is formed from material selected from the group comprising polyurethane, polystyrene, oxycellulose, and polyvinylalcohol.

6. A bar element in accordance with claim 1 wherein said core is hollow.

7. A bar element in accordance with claim 1 wherein said core is solid.

8. A bar element in accordance with claim 1 wherein said core is buoyant.

9. A bar element in accordance with claim 1 wherein said soap is selected from the group consisting of shop or lavatory hand soap, deodorant soap, antibacterial soap, bath soap, hair soap, facial soap, cosmetic soap, and any combinations thereof.

10. A bar element in accordance with claim 1 wherein said element is formed by being severed from a longer bar element having a continuous core and a continuous soap shell.
